(19) United States
(12) Patent Application Publication
(10) Pub. No.: US 2005/0180927 A1
(43) Pub. Date: Aug. 18, 2005

(54) Novel anti-bacterial compositions

(76) Inventors: Graham Leslie Aldous, Adelaide (AU); Richard Scott Blake, Adelaide (AU); Michael Scott Blake, Adelaide (AU)

Correspondence Address: KLAUBER & JACKSON, 411 HACKENSACK AVENUE, HACKENSACK, NJ, 07601, US

(21) Appl. No.: 10/509,811

(22) PCT Filed: Apr. 4, 2003
(86) PCT No.: PCT/AU03/00410

(30) Foreign Application Priority Data
Apr. 4, 2002 (AU) PS 1532

(51) Int. Cl. A61K 7/16

(52) U.S. Cl. 424/49

(57) ABSTRACT

The oral formulations of the present invention comprise: (a) chlorhexidine or a salt thereof; (b) a zinc salt; (c) masking and/or flavouring agents, including: (i) a sweetening agent having an immediate but transient effect and (ii) a sweetening agent having a delayed but prolonged effect; and (d) other conventional components of oral formulations. The formulations are, for example, toothpastes, dentifrices, mouthwashes, chewing gum or lozenges.

### BACKGROUND TO THE INVENTION

The present invention relates to novel formulations for toothpastes, dentifrices, mouthwashes, chewing gum or lozenges, which may be used in the treatment of oral health problems, such as dental plaque, gingivitis and dental calculus, or as part of everyday oral hygiene practice.

Dental plaque is a complex mass, consisting mainly of bacteria that colonise the dental pellicle, the metabolic products of those bacteria, and other cellular material (epithelial cells and leukocytes). Dental plaque is the main etiological factor responsible for caries and periodontal diseases. The number and nature of the bacteria change continuously as the plaque develops, and different sites in the mouth may host different bacterial populations. The mode of attachment or aggregation of much of the oral bacteria is unclear. However, salivary aggregation, direct inter-species attachment, secretion of extra-cellular polysaccharides, physical entrapment of organisms, and presence of site-specific receptors are important factors.

The structure of plaque is soft and readily disrupted. The plaque structure derives from the properties of bacterial cell walls, cell wall polymers, and electrostatic bonding based on the presence of divalent cations such as calcium. Gingivitis is caused when bacteria begin to grow at the gingival margin, generating toxins that cause inflammation. Gingivitis may be recognised by the gums becoming red and puffy, bleeding of the gums when they are subjected to minor trauma as caused by a toothbrush or flossing, and persistent bad breath. Unless treated, gingivitis can progress to periodontal disease. Although the progress from gingivitis to periodontal disease is not fully understood, it is believed that the process may follow the following stages:
bacterial growth at the gingival margin;
inflammation and bleeding of the gums;
formation of gingival pockets (sulci);
deepening of the gingival pockets to form periodontal pockets;
trapping of plaque micro-organisms, debris and other material in these pockets;
degenerative changes in surrounding connective tissue, caused by cellular and
fluid exudates;
widening of periodontal pockets, causing loosening of the teeth; and
receding of the gums, thus exposing the roots of the teeth and causing
discomfort.

Dental calculus is calcified plaque. Once considered to be the primary cause of periodontal disease through irritation, calculus is now considered to be of secondary importance. Nevertheless, calculus can be viewed as being the substrate on which further plaque can form.

Supragingival calculus arises from nucleation and subsequent crystallization of calcium phosphate in plaque.

The aim of the present invention is to produce a toothpaste, dentifrice, mouthwash, chewing gum or lozenge which is pleasant tasting and has a good mouth-feel, is low staining, cleans teeth and significantly reduces plaque build-up.

An ideal anti-plaque agent would inhibit bacterial adhesion to oral surfaces, disrupt pre-formed bacterial masses such as plaque, and maintain its effect for a long period of time.

Chlorhexidine or salts thereof are the most effective chemical anti-plaque agents currently available.

Chlorhexidine or salts thereof are highly effective in reducing oral plaque-forming bacteria associated with tooth decay, and gingivitis, and are therefore a particularly effective (and safe) agent in the treatment of oral health problems. Animal and human studies have demonstrated that chlorhexidine, or a salt thereof, in a mouthwash can effectively inhibit formation of dental plaque and gingival disease.

The mechanism of action of chlorhexidine (or salts thereof is multifaceted:
It interferes with bacterial absorption to teeth;
has anti-plaque activity at very low concentrations;
absorbs onto mucous membranes and teeth, desorbing from oral surfaces over a period of days;
has a long-lasting action, with residual anti-bacterial effects as long as 24 hours after application;
is effective against a wide range of Gram-positive and Gram-negative bacteria; and
leads to a general reduction of salivary microflora, including yeast and fungi.

The exceptional anti-plaque activity of chlorhexidine or salts thereof can be attributed to their ability to adsorb onto dental surfaces and desorb therefrom gradually, providing, in effect, a timed release of the anti-microbial agent.

The anti-microbial properties of the chlorhexidine or salt thereof, which is the active ingredient of the toothpastes, dentifrices, mouthwashes, chewing gum and lozenges of the present invention, is believed to derive from the following:

At high concentrations, it acts as a detergent, acting on cell membranes and causing loss of cytoplasmic constituents; at concentrations equal to or below the Minimum Inhibitory Concentration (MIC), it inhibits membrane transportation, metabolism and the activity of membrane-bound ATPase and various other enzymes.

However, there are serious problems in using chlorhexidine or its salts in oral hygiene products, such as toothpastes, dentifrices, mouthwashes, chewing gum and lozenges. Although considered safe for oral use, chlorhexidine and its salts are not widely used in toothpastes, dentifrices, mouthwashes, chewing gum and lozenges because they:
stain teeth, gums, the tongue and oral mucosa,
have an exceptionally bitter, long-lasting and unpleasant taste, and
are bio-inactivated in the presence of a range of common toothpaste ingredients.

The first two problems (staining of teeth and bitter taste) have meant that chlorhexidine and its salts have failed to gain widespread acceptance because of consumer rejection.

The mechanism of staining is not well understood, but is known to be influenced by factors in the diet (such as tea, coffee or red wine) or personal habits (such as smoking).

The patent literature includes many examples of overcoming each of these individual side-effects, but no example of a composition which can overcome all three side-effects simultaneously.

The use of zinc salts in dentifrices has been widely reported in the literature, and they are now found in many dentifrices and mouthwashes marketed worldwide. The salts have mainly been added for their astringent properties to treat ulcerated, abraded or inflamed oral surfaces.

Zinc salts, along with a variety of heavy metals (eg copper and nickel), are well-known for their anti-plaque properties, and reportedly have a synergistic effect when used in conjunction with anti-microbials. The ability of Zn²⁺ salts to inhibit the development of dental calculus has also been demonstrated. The effect of Zn²⁺ ions appears to be mainly attributable to competition with Ca²⁺, thereby producing structural defects in the plaque-calculus formation system that make the system less resistant to mechanical forces such as salivary lavage and dental hygiene, and more amenable to penetration by anti-microbial agents. In general, the zinc salts used in oral preparations are of limited solubility, but a zinc complex of gluconic acid (ie zinc gluconate) is soluble in water and is ideal for such preparations.

The use of zinc salts or other methods, to prevent staining of the oral cavity by chlorhexidine, has also been reported (see, for example, J. Clinical Periodontology (1994), 2, 431-443). However, the product either has failed to taste satisfactory or has been bio-inactivated or both.

Similarly, a pleasant tasting product may cause unacceptable staining, be bio-inactivated or both, and a bio-active product may cause staining or have unacceptable taste characteristics.

The use of cyclodextrins to mask bitter flavours has been the subject of many patents in USA and elsewhere. In particular, Warner-Lambert (AU-B-20058/88; EP-306455A; JP 1090165A) discloses the use of cydodextrin complexes to mask the bitter taste of chlorhexidine, and enhance its bioavailability.

Sodium saccharin and chlorhexidine combine to form a salt which is only slightly soluble in water (JP 004891 (1963)), and its use as an anti-plaque agent is disclosed in U.S. Pat. No. 4,614,649. However, the insolubility of the salt limits its use. In particular, addition of chlorhexidine gluconate (for example) to a dentifrice formulation which also comprises sodium saccharin leads to precipitation of the chlorhexidine saccharinate salt and reduction in the bioactivity of the chlorhexidine.

Another problem is that the sweetening effect of saccharin rapidly diminishes in the mouth, whereas the bitter taste of chlorhexidine is detectable long after it is first tasted. This is due to the binding of chlorhexidine to oral surfaces and its subsequent slow release over a period of days.

Neohesperidine dihydrochalcone is a natural sweetener that is three to ten times sweeter than saccharin (ie as much as 2,000 times sweeter than sugar), but has the problem that the sweetening effect is delayed, so impractically large quantities of the compound must be used to provide an initial sweetening effect in the presence of bitter tasting compounds—after a short delay, this leads to over-sweetening.

The main challenge therefore is to produce a chlorhexidine-containing oral formulation, such as a toothpaste, a dentifrice, mouthwash, chewing gum or a lozenge, that is bioactive in terms of anti-plaque and anti-gingival effects; pleasant tasting with acceptable mouth-feel; and with staining due to the chlorhexidine significantly reduced, down to acceptable levels. This is achieved by the novel formulations of the present invention.

### SUMMARY OF THE INVENTION

The oral formulation of the present invention comprises:
a) chlorhexidine or a salt thereof, such as chlorhexidine digluconate (commonly known as chlorhexidine gluconate), chlorhexidine diacetate or chlorhexidine dihydrochloride;
b) a zinc salt, such as zinc gluconate;
c) masking and/or flavouring agents, including (i) a sweetening agent having an immediate but transient effect (for example, for a matter of minutes after administration), such as saccharin or a salt thereof, and (ii) a sweetening agent having a delayed but prolonged effect, such as neohesperidine dihydrochalcone; and
d) other conventional components of oral formulations.

The oral formulation may be a solution of the components, such as mouthwash; a semi-solid product, such as toothpaste or gel dentifrice; chewing gum; or a solid lozenge.

Preferably, the chlorhexidine or salt thereof forms 0.1 to 1.0% w/w of the formulation, and the zinc salt similarly forms 0.1 to 1.0% w/w of the formulation.

Besides the sweetening agents referred to in c) above (eg saccharin, or a salt thereof, and neohesperidine dihydrochalcone), the oral formulation of the present invention may also comprise additional masking and/or flavouring agents. Examples of suitable masking and/or flavouring agents include, but are not limited to, flavouring oils (eg oil of spearmint, peppermint, wintergreen, sassafras, dove, sage, eucalyptus, cinnamon, lemon and orange) and methyl salicylate.

The use of masking and/or flavouring agents is necessary since conventional sweetening agents (such as xylitol and sorbitol) are not sufficient to cover the bitter chlorhexidine taste. The taste is persistent and previous attempts to mask it in oral preparations containing chlorhexidine have had variable success. Artificial sweetening agents such as saccharin and its salts and cyclamate and its salts have been used. It is known, however, that saccharin (and salts thereof) will complex with chlorhexidine (and salts thereof), causing precipitation leading to bio-inactivation of the chlorhexidine. Also, the masking effect of such artificial sweeteners is transient, lasting only a short time while the masking compound is present in the mouth. Thus, saccharin, or a salt thereof, on its own is ineffective. Neohesperidine dihydrochalcone, being of medium intensity sweetness, is persistent in the long term but does not mask in the short term the immediate bitter taste of the chlorhexidine, and is also ineffective on its own.

Surprisingly, it has been discovered that certain combinations of saccharin or a salt thereof, preferably saccharin sodium, with neohesperidine dihydrochalcone did not bio-inactivate or precipitate the chlorhexidine, and yet provided a synergistic long-lasting masking effect of the persistent bitter chlorhexidine taste. The preferred maximum level of saccharin sodium to be used in the presence of neohesperidine dihydrochalcone (which has a preferred maximum level of 0.1% w/w, and more preferably up to 0.05% w/w), and in the presence of anti-plaque effective concentrations of chlorhexidine or its salts, was 0.05% w/w.

The efficacy of this combination of sweetening agents is particularly surprising in that saccharin and its salts are contra-indicated for use as sweetening agents in chlorhexidine-containing oral formulations. The relatively high concentrations necessary when saccharin or a salt thereof is the sole sweetening agent lead to precipitation of the chlorhexidine and reduced bioactivity.

The preferred maximum level of neohesperidine dihydrochalcone to be used in the oral formulations of the present invention, in the presence of those same levels of chlorhexidine and saccharin salts (ie anti-plaque effective concentrations and up to 0.05% w/w, respectively), was 0.05% w/w, higher levels leading to an unpleasant level of sweetness.

When combined with one or more of the flavouring oils described above, the product produced was pleasant tasting with little after-taste. Accordingly, the masking and/or flavouring agents of the present invention preferably include saccharin or a salt thereof and neohesperidine dihydrochalcone. Suitable flavouring and sweetening agents may each or together comprise from about 0.1% to 5% w/w or more of the preparation. The use of a combination of relatively low levels of saccharin or a salt thereof with a long-lasting sweetener being neohesperidine dihydrochalcone, as a sweetening/masking agent in chlorhexidine formulations, forms a novel aspect of the present invention.

Surfactants may be used in the compositions of the present invention to achieve increased foaming action and maintain the flavours in dispersion. The surfactant material most commonly used in toothpaste is anionic; however, this class of surfactant is incompatible with chlorhexidine and its salts. According to one aspect of the present invention, a combination of non-ionic and zwitterionic surfactants is used, which combination provides good foaming of the toothpaste and does not bio-inactivate the chlorhexidine. Typical non-ionic surfactants are, for example, macrogol ethers (condensation products of polyethylene glycol and fatty alcohols, usually cetyl or cetylstearyl alcohol). Typical zwitterionic surfactants are, for example, betaines of general structure RN⁺(C)₂CH₂COO— and alkylamido alkyl amines having a general formula of, for example, RCONH(CH₂)₃N⁺(CH₃)₂CH₂COO—. A suitable combination is of the macrogol ether, ceteareth 30, and cocamidopropyl betaine, eg in the ratio of 2.4:1 by weight, which may, for instance, constitute a total of 1.7% w/w of the toothpaste, although the maximum content of the combination may be as high as 10% w/w and as low as 0.1% w/w.

When the oral composition is substantially semi-solid or pasty in character, such as a toothpaste or gel, the dentifrice vehicle may contain a dentally acceptable water insoluble abrasive material such as sodium metaphosphate, potassium metaphosphate, tricalcium phosphate, dihydrated dicalcium phosphate, anhydrous dicalcium phosphate, calcium pyrophosphate, calcium carbonate, aluminum silicate, hydrated alumina, calcined alumina, silica, bentonite, and mixtures thereof.

The abrasive material is generally present in the paste or gel composition in weight concentrations of about 5% to about 60% by weight, preferably about 10% to about 30% in a gel and about 5% to about 60% in a paste.

Toothpastes, as well as gel dentifrices, typically contain a natural or synthetic thickener or gelling agent in proportions of about 0.1 to about 10% by weight, preferably about 0.5 to about 5% by weight. Suitable thickeners or gelling agents include Irish moss, carrageenan, gum tragacanth, starch, polyvinylpyrrolidone, hydroxyethyl propyl cellulose, hydroxybutyl methyl cellulose, hydroxypropyl methyl cellulose, hydroxyethyl cellulose, Veegum and other silica-derived materials.

Fluoride materials may also be included in the oral compositions of the present invention to provide an anti-caries effect. Suitable such materials are inorganic fluoride salts, preferably soluble alkali metal fluoride salts, for example sodium fluoride, potassium fluoride, sodium monofluorophosphate and sodium hexafluorosilicate. The fluoride-providing salt is generally present in the oral composition at a concentration of about 0.0005 to about 3.0% by weight.

Where the oral composition of the present invention is substantially liquid in character, such as a mouthwash or rinse, the vehicle is typically a water-alcohol mixture. Generally, the weight ratio of water to alcohol is in the range of from about 3:1 to 10:1, and preferably about 4:1 to about 6:1. The alcohol is a non-toxic alcohol such as ethanol or isopropanol. A humectant, such as glycerine, sorbitol or an alkylene glycol such as polyethylene glycol or preferably propylene glycol, may be present in an amount of about 10-30% by weight. Mouthwashes or rinses typically contain about 50-85% by weight of water, about 0 to 20% by weight of a non-toxic alcohol and about 10-40% by weight of the humectant.

A toothpaste formulated according to the present invention, and comprising 0.6% (w/w) chlorhexidine digluconate, has the following properties:
it is biologically active and chemically stable;
it is pleasant tasting;
it exhibits low chlorhexidine induced staining;
it significantly reduces plaque build-up; and
it cleans teeth, leaving a good mouth feel.

The present invention provides a method of incorporating chlorhexidine or a salt thereof in an oral formulation (eg a toothpaste, dentifrice, mouthwash, chewing gum or lozenge), so as to form a product having acceptable characteristics with regard to bioactivity, taste and staining of teeth, wherein the chlorhexidine or salt thereof is accompanied by a zinc salt, and the toothpaste, dentifrice, mouthwash, chewing gum or lozenge further comprises: masking and/or flavouring agents including (i) a sweetening agent having an immediate but transient effect, such as saccharin or a salt thereof, and (ii) a sweetening agent having a delayed but prolonged effect, such as neohesperidine dihydrochalcone; and other conventional ingredients of dental products, including surfactants, preservatives, thickeners etc.

### DETAILED DESCRIPTION OF THE INVENTION

### EXAMPLES

The invention will now be further described with respect to the following examples, which are illustrative but not restrictive of the present invention.

### Example 1

### Toothpaste Formulation

A typical toothpaste formulation according to the present invention is prepared as described below:

| | % | |
|---|---|---|
| COMPONENT | w/w | |
| ALCOHOL 95% | 7.5-10 | 1 |
| FUMED SILICA | 0.8-5.0 | 2 |
| COCAMIDOPROPYL BETAINE | 0.5 | 3 |
| CETEARETH 30 | 1.2 | 4 |
| CHLORHEXIDINE DIGLUCONATE SOLUTION | 3.195 | 5 |
| 20% W/V | | |
| HYDRATED SILICA | 5-20 | 6 |
| HYDROXY ETHYLCELLULOSE | 0.5-5.0 | 7 |
| METHYL para HYDROXYBENZOATE | 0.1 | 8 |
| METHYL SALICYLATE | 0.15 | 9 |
| NEOHESPERIDINE DIHYDROCHALCONE | 0.01-0.1 | 10 |
| PROPYL HYDROXY BENZOATE | 0.05 | 11 |
| SACCHARIN SODIUM | 0.01-0.05 | 12 |
| SORBITOL SOLUTION 70% | 45.0 | 13 |
| WATER PURIFIED | qs 100 | 14 |
| XYLITOL | 2.5 | 15 |
| ZINC GLUCONATE | 0.1-1.0 | 16 |
| PEPPERMINT OIL | 0.4 | 17 |

Method

A. Dissolve, with stirring, components 3, 4, 8, 9, 10, 11, 12 and 17 in alcohol 1. Finally add component 7 and continue to stir.

B. To an appropriate quantity of purified water 14 add components 15,16 and then 13. Stir to dissolve, and then add component 5. Continue stirring until a clear solution is obtained.

C. To the above solution B add components 2 and 6 and mix well with gentle stirring until a uniform dispersion is obtained. Finally add phase A with stirring. Allow to thicken.

### Example 2

### Toothpaste Formulation

The formulation is similar to that of Example 1, except that 0.01-0.22% w/w of sodium fluoride is added to the aqueous phase B.

### Example 3

### Clinical Studies

Introduction

A study was carried out in the Department of Dentistry, University of Adelaide, in conjunction with Hamilton Laboratories, to test the effect of a new formulation of a toothpaste containing chlorhexidine (CHX) gluconate (as per Example 1) on dental plaque formation. Slurries of the toothpastes being tested were prepared and used as rinses, with subjects abstaining from mechanical plaque control for 4 days. The slurry method is used because mechanical plaque control alone, performed by a skilled person, would reduce dental plaque formation, with or without the use of a toothpaste or antibacterial agent. A commercial Oral Rinse (mouthwash) formulation from Zila Inc (Peridex, containing 0.12% w/w chlorhexidine gluconate solution) was chosen as a positive control because of its well documented ability to suppress plaque formation.

Methods

Subjects

Volunteers participating in the study were non-smokers, in good general health and included both women and men between 20 and 55 years of age. They did not use any mouthwash or toothpaste containing chlorhexidine as part of their routine oral hygiene practices. The six subjects were chosen because they formed large amounts of plaque in a previous pilot study.

The following protocol was used:

The trial was conducted over a period of several weeks, with the subjects attending a dental clinic on the morning of the first day for a prophylaxis. The subjects were then provided with one of four blinded samples to be used as a rinse on the evening of day 1; twice daily, morning and evening, for days 2,3,4; and once in the morning of day 5. Mechanical plaque control was not allowed during this period. On the morning of day 5, the subjects again attended the clinic, the plaque on their teeth was disclosed by the application of a dye, and a score in the range of 0-5 (where 0 was no plaque, 5 was maximum plaque) for individual teeth was determined. The teeth were then photographed and given a prophylaxis. The subjects were then allowed to resume their habitual plaque control for a week while resting, to allow for washout of any chlorhexidine they may have encountered. On the first day of the third week, the whole process was repeated, with the subject receiving a different preparation to trial, and so on until all four samples had been tested. One of the randomized samples was a chlorhexidine mouthwash used as a control. This control has been widely used in clinical trials examining the anti-plaque activity of toothpastes as it provides a consistent, very high anti-plaque activity, but it is a simple non-viscous solution without the complexity of taste, foaming and physical characteristics expected of a consumer product.

At no time did the subject or the examiner know which preparation was being used. The four preparations were randomly distributed amongst the subjects in such a manner that all subjects used all four preparations but in varying order. The key was held in a secure location until the completion of the trial.

The protocol is summed in Table 1.

TABLE 1

| Monday | Friday |
|---|---|
| Prophylaxis. | Plaque disclosing. |
| Cease mechanical plaque control. | Photograph anterior teeth (⅔ |
| | magnification). |
| Commence rinsing 2 × daily for 60 secs | Prophylaxis. |
| with 3 g toothpaste slurry. | |
| | Recommence habitual plaque |
| | control. |

The samples used were:
A-3 g of Hamilton CHX toothpaste (0.6% w/w) with Fluoride, mixed in a 15 ml slurry
B-3 g of Hamilton CHX toothpaste (0.6% w/w), mixed in a 15 ml slurry
C-15 ml of Chlorhexidine Gluconate solution (0.12% w/w solution) (Control-Peridex mouthwash)
D-3 g of Placebo toothpaste, mixed in a 15 ml slurry

Preparations A to C each contained 18 mg of chlorhexidine (CHX).

Determination of Plaque Coverage of Teeth

The teeth were examined after 4 days, and a dye was applied to highlight the plaque. A photograph of the teeth was taken as a record.

Results

Assessment of Plaque Area

The plaque indexes for the four preparations were determined.

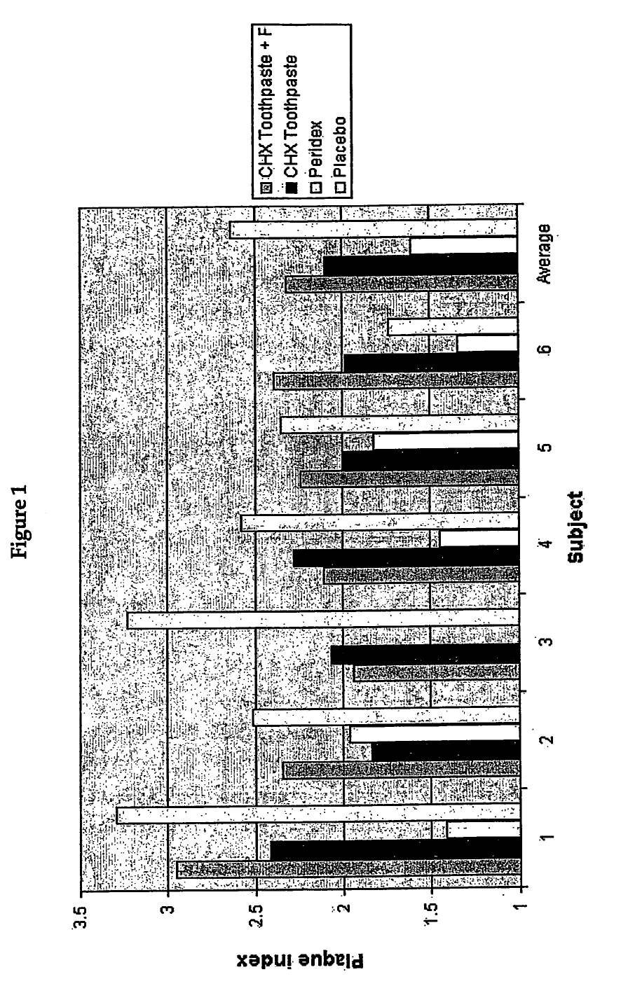

FIG. 1 shows plaque indices for the six subjects, as assessed after four days. The plaque scores were taken using the Turesky modification of the Quigley-Hein index (1-5 scale).

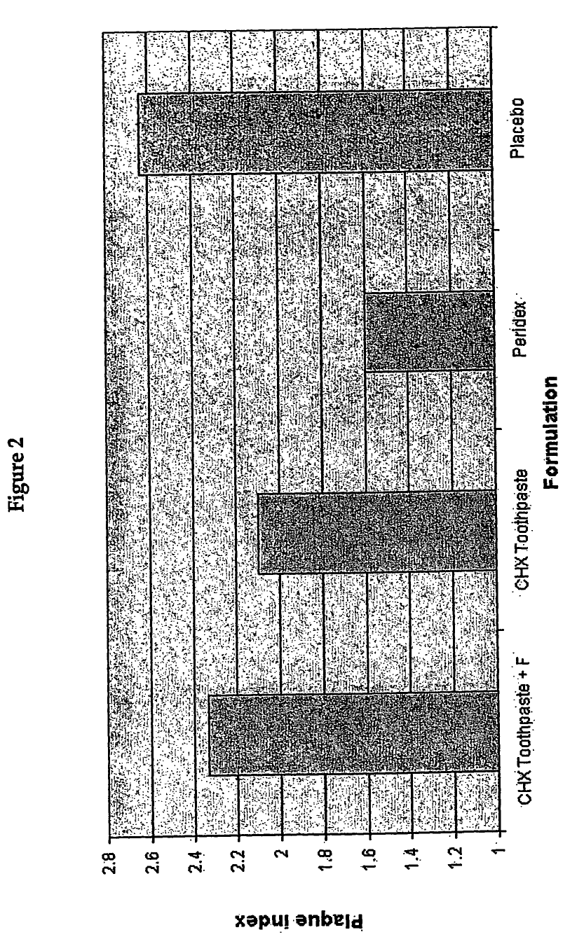

FIG. 2 shows the average results of the six subjects.

The control mouthwash, Preparation C, provided the anticipated level of plaque protection. There was a mathematically significant difference (p<0.05) between Preparation B (0.6% chlorhexidine gluconate toothpaste) and the placebo (Preparation D), and also between Preparation A (0.6% chlorhexidine gluconate toothpaste with fluoride) and the placebo (Preparation D)—Preparations A and B both performed significantly better than the placebo. Although plaque suppression by Preparation B was better than for Preparation A, Preparation A still provided acceptable results.

There was also a mathematically significant difference (p<0.05) between Preparation B and Preparation C (Peridex mouthwash).

Preparations A and B both performed midway between the placebo (Preparation D) and Peridex mouthwash (Preparation C).

The toothpaste slurries (Preparations A and B; also, the control Preparation D) used in the trial are, by their nature, more viscous and more chemically complex than the mouthwash (Preparation C). These factors lead to longer diffusion times of the CHX from the slurry and onto the oral surfaces. Taking these factors into consideration, Preparations A and B performed very well in comparison to Peridex (Preparation C). Staining was visually assessed. The staining caused by Preparations A and B was found to have limited distribution in the mouth (lingual or lower incisors), and was relatively easily removed as compared to conventional chlorhexidine staining. This indicated that Preparations A and B did not have a significant staining effect, whereas staining is a very real problem when using conventional chlorhexidine products.

While the present invention has been described in terms of preferred embodiments in order to facilitate better understanding of the invention, it should be appreciated that various modifications can be made without departing from the principles of the invention. Therefore, the invention should be understood to include all such modifications within its scope.

1-24. (canceled)

25. An oral formulation comprising:
(a) chlorhexidine or a salt thereof;
(b) a zinc salt;
(c) masking and/or flavouring agents, including (i) a first sweetening agent having an immediate but transient effect and (ii) a second sweetening agent having a delayed but prolonged effect, said second sweetening agent being neohesperidine chalcone; and
(d) other conventional components of oral formulations.

26. An oral formulation according to claim 25, wherein said first sweetening agent is saccharin or a salt thereof.

27. An oral formulation according to claim 26, comprising up to 0.05% (w/w) of saccharin sodium.

28. An oral formulation according to claim 25, comprising up to 0.1% (w/w) of neohesperidine dihydrochalcone.

29. An oral formulation according to claim 25, comprising 0.1 to 1.0% (w/w) of chlorhexidine or a salt thereof.

30. An oral formulation according to claim 25, comprising 0.1 to 1.0% (w/w) of the zinc salt.

31. An oral formulation according to claim 25, comprising one or more gluconate salt(s).

32. An oral formulation according to claim 25, wherein the zinc salt is zinc gluconate.

33. An oral formulation according to claim 25, wherein the chlorhexidine salt is chlorhexidine digluconate.

34. An oral formulation according to claim 33, comprising about 0.6% (w/w) of chlorhexidine digluconate.

35. An oral formulation according to claim 25, wherein the chlorhexidine salt is chlorhexidine diacetate.

36. An oral formulation according to claim 25, further comprising additional masking and/or flavouring agents selected from flavouring oils and methyl salicylate.

37. An oral formulation according to claim 25, comprising 0.1 to 5% (w/w) of said masking and/or flavouring agents.

38. An oral formulation according to claim 25, comprising components (d) selected from the group consisting of: fluoride materials, dentally acceptable abrasive materials, surfactants, thickeners, gelling agents, humectants, alcohol and water.

39. An oral formulation according to claim 38, wherein said surfactants are selected from non-ionic and zwitterionic surfactants.

40. An oral formulation according to claim 39, wherein said non-ionic surfactants are macrogol ethers.

41. An oral formulation according to claim 39, wherein said zwitterionic surfactants are selected from the group consisting of betaines and alkylamido alkyl amines.

42. An oral formulation according to claim 39, wherein said surfactants comprise a combination of non-ionic and zwitterionic surfactants.

43. An oral formulation according to claim 42, wherein said surfactants comprise a combination of a macrogol ether and cocamidopropyl betaine.

44. An oral formulation according to claim 42, wherein the ratio of the non-ionic surfactant(s) to the zwitterionic surfactant(s) is about 2.4:1 by weight.

45. An oral formulation according to claim 39, comprising 0.1 to 10% (w/w) of said surfactants.

46. An oral formulation according to claim 45, comprising about 1.7% (w/w) of said surfactants.

47. An oral formulation according to claim 25, being selected from the group consisting of toothpastes, dentifrices, mouthwashes, chewing gum and lozenges.
